(19) United States
(12) Patent Application Publication
(10) Pub. No.: US 2026/0108435 A1
(43) Pub. Date: Apr. 23, 2026

(54) SUNSCREEN COMPOSITION WITH FUNCTIONALIZED ZEOLITE SPF BOOSTERS

(71) Applicant: SAES GETTERS S.P.A., Lainate MI (IT)

(72) Inventors: Miriam RIVA, Lomazzo CO (IT); Katarzyna FIDECKA, Cantù CO (IT); Agnello DE SIMONE, Saronno VA (IT)

(73) Assignee: SAES GETTERS S.P.A., Lainate MI (IT)

(21) Appl. No.: 19/487,541

(22) PCT Filed: Apr. 4, 2025
(86) PCT No.: PCT/EP2025/059380

§ 371 Date: Nov. 25, 2025

(30) Foreign Application Priority Data
Apr. 5, 2024 (IT) 102024000007582
Jul. 22, 2024 (IT) 102024000016930

(51) Int. Cl. A61K 8/26; A61Q 17/04

(52) U.S. Cl. CPC A61K 8/26; A61Q 17/04; A61K 2800/412; A61K 2800/5922; A61K 2800/612; A61K 2800/651

(57) ABSTRACT

The present invention relates to sunscreen compositions for protecting damage from ultraviolet (UV) rays, comprising SPF boosters based on specific zeolites functionalized with a silane moiety.

### FIELD OF INVENTION

The present invention relates generally to sunscreen compositions for protecting damage from ultraviolet (UV) rays. More specifically, the invention is direct to a sunscreen formulation with SPF boosters based on specific zeolites functionalized with a silane moiety.

### DESCRIPTION

The negative effects of exposure to ultraviolet (UV) light having a wavelength of from about 200 nm to about 400 nm, are generally recognized. The prolonged non-shielded exposure to solar radiation causes adverse health consequences, such as the immediately appearing of painful sunburns, and long-term damage which can lead to serious conditions such as skin cancer.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products.

In order to reduce the amount of solar UV radiation received by the skin during exposure to the sun radiation, different sunscreen compositions can be used.

A sun care composition may contain inorganic UV filters, also called physical filters, and/or chemical UV filters which are organic molecules. The inorganic UV filters interact with UV light by two mechanisms: absorption and reflection/scattering, while the organic filters typically contain aromatic carbon and/or other electron-dense bonds that are responsible for absorbing light in the UV ranges of the solar spectrum.

The higher the amount of UV filters, the greater the degree of UV protection, however, it has recently become evident that too high concentration of both inorganic and organic UV filters, not only compromise the sun care formulation aesthetics, but it also brings unfavorable safety impacts on both human health and the environment.

Therefore, a key challenge that arose in this field is represented by the reduction of the quantities of UV filters used in sunscreen formulations while still ensuring high sun protection efficacy.

According to that, an effective strategy to solve this problem involves the use of a sun protection factor (SPF) booster into sunscreen formulations. SPF boosters are defined as compounds, safe for both human use and the environment, which are not recognized as sunscreen actives, but work to increase the SPF of the composition in which they are incorporated.

Typical sunscreen formulations are emulsions, serums, creams, or gels containing, in addition to chemical and/or physical UV filters, many other compounds such as emulsifiers, solubilizers, stabilizers, preservatives and also SPF boosters; all of which influence the protectiveness of the sunscreen, the activity of the SPF Booster and the and cosmetic appeal.

As reported in WO2017112982 and WO2021102873 a widely used inorganic SPF booster is represented by hydrophobic silica particles, also in the form of silica particles whose surface is treated with silylating agents. U.S. Pat. No. 9,265,715 claims for example the use of silica particles in combination with nylon and barium sulfate particles in order to have a boosting effect on sunscreen composition. A further example is represented by US20210330571 in which hydrophobic fine particulate of titanium oxide or zinc oxide, ensure a boosting effect to the final composition. Alternatively, WO2022081942 describes an organic compound, specifically xanthommatin, as SPF booster in combination with a chemical UV filter.

However, some of the most cited classes of compounds such as silica has been progressively reduced due to their toxicity for human and environment. While inorganic compounds as titanium oxide and zinc oxide or organic compounds as the xanthommatin above reported, cannot be considered an SPF booster since they act as a real UV filter (the so-called SPF-doping effect).

Thus, object of the present invention is a sunscreen composition comprising at least one UV filter and at least a specific type of zeolite selected among Beta type zeolite, ZSM-5 type (MFI), Linde type (LTA), Mordenite type (MOR) or Faujasite type (FAU) functionalized with a silane moiety selected among thiethoxycaprylyl silane Stearyl Triethoxysilane, Trihydroxymethylsilane and Methoxypolyethyleneoxypropyltrymetoxysilane

In particular, the inventors found that said specific functionalized zeolites, which do not show a sunscreen activity as a single component, act as an efficient SPF booster when formulated in a sunscreen formulation comprising UV filters, thus avoiding the SPF doper effect according to the international safety regulation.

Moreover, it has been clearly pointed out that, the functionalization of said zeolites and the consequent amphiphilic nature acquired, improves the dispersibility of the same in both one-phase and two-phase formulations and reduces the aggregation phenomena in the final formulation with a consequent significant increase of the SPF Boosting effect. Indeed, as reported in the below experimental part, the boost ensured by said specific zeolites to the final composition SPF is at least 20%.

Said SPF boosters have also been proved to be compatible both with marine friendly UV filters and other marine friendly sunscreens' components, and with water resistant components for water resistant sunscreen formulations.

In a preferred embodiment, the zeolites are characterized by the presence of ammonium or hydrogen as counter-ion at an ion exchange site of the framework structure with a pH comprise in a range between 3.0 and 8.0.

In a further aspect, the zeolites are characterized by a particle size distribution is comprised between 0.1 and 15 μm, preferably between 0.2 to 5.0 μm.

The functionalization of the zeolites are made with a silane selected among thiethoxycaprylyl silane Stearyl Triethoxysilane, Trihydroxymethylsilane or Methoxypolyethyleneoxypropyltrymetoxysilane in an amount comprised between 0.2 and 10.0%, preferably between 2.0 and 6.0% with respect to the total weight of the zeolite.

According to the invention, the amount of zeolites is comprised between 0.1 and 5.0% wt, preferably between 1.0 and 3.0% wt with respect to the total weight of the composition; while the amount of the UV filter is comprised between 1.0 and 35% wt with respect to the total weight of the composition.

In a preferred embodiment, specifically when an organic UV filter is employed, the amount of said compound is comprised between 1 and 15% wt with respect to the total amount of the composition, while the amount is preferably comprised between 1 and 35% wt with respect to the total amount of the composition, when said filter is an an inorganic compound.

Indeed, the UV filters employed in the sunscreen composition described herein can be an organic molecule, also defined as chemical filter, an inorganic compound, also defined as physical filter or mixture thereof.

Said organic molecules are selected in a group consisting of Octocrylene, Ethylhexyl Salicylate, Homosalate, Butyl Methoxydibenzoylmethane, Diethylamino hydroxybenzoyl hexyl benzoate, Bis-Ethylhexyloxyphenol Methoxyphenyl Triazine, Ethylhexyl Triazone, Avobenzone, Diethylhexyl Butamido Triazone, Ethylhexyl Methoxycinnamate, Phenylbenzimidazole sulfonic acid, Tris-biphenyl triazine, Tris-biphenyltrazine (nano), Oxybenzone, Octinoxate, Octyltriazone, Octisalate, Padimate O, Bis-piperazine HAA299, Bis-piperazine HAA299 (nano), (2-Ethoxyethyl (2Z)-2-cyano-2-[3-(3-methoxypropylamino) cyclohex-2-en-1-ylidene] acetate), Sulisobenzone, Drometrizole trisiloxane, Methylene bis-benzotriazolyl tetramethylbutylphenol, Benzylidene camphor sulfonic acid, 4-Methylbenzylidene Camphor, Polysilicone-15, Sulisobenzone sodium, Aminobenzoic acid, PEG-25 PABA, Methylene Bis-Benzotriazolyl Tetramethylbutylphenol, Methylene Bis-Benzotriazolyl Tetramethylbutylphenol nano, Menthyl anthranilate, Isopentyl-4-methoxycinnamate, terephthalylidene dicamphor sulfonic acid, Dioxibenzone, Cinoxate, camphor benzalkonium sulfate and mixtures thereof possibly considering all size ranges both nano-, sub- and micro-metric, including modification by any means (via chemical or physical approaches).

While the inorganic compounds are selected among Zinc Oxide, Anatase titanium dioxide, Rutile, titanium dioxide and mixtures thereof possibly considering all size ranges both nano-, sub- and micro-metric.

As reported above, the SPF boosters herein described are extremely versatile and compatible in several matrixes, thus according to the present invention, the final sunscreen composition can be formulated either as a single-phase formulation, such as a gel or a stick, or as a two-phase formulation such as an homogenous oil-in-water emulsion, such as a serum or a cream.

The sunscreen composition according to the invention can also comprise at least one of a cosmetically acceptable emollient, carrier fluid, pigment, humectant, vitamin, antioxidant, emulsifier, co-emulsifier, hydrophilic or hydrophobic thickeners, wax, butter, film former, silicone, surfactant, active agent, extract, fragrance, preservative, pH-adjusting agent, suspending agent, chelating agent.

The present invention also refers to the composition according to any of the embodiment herein disclosed as a sunscreen composition or agent. In particular, the invention refers to a composition according to any of the embodiment herein disclosed for use in a method for protecting a keratinous surface (e.g. skin) from UV radiation comprising contacting the keratinous surface with the sunscreen composition of the invention.

The invention also refers to the composition as herein disclosed as an additive or adjuvant to a sunscreen composition. In addition, the invention refers to the zeolites or functionalized zeolites as disclosed herein, particularly selected in a group consisting of Beta type, ZSM-5 type (MFI), Linde type (LTA), Mordenite type (MOR) and Faujasite type (FAU) functionalized with a silane moiety selected among thiethoxycaprylyl silane Stearyl Triethoxysilane, Trihydroxymethylsilane or Methoxypolyethyleneoxypropyltry-metoxysilane, for use as adjuvant in sunscreen compositions or for use in boosting the SPF effect of UV filters in sunscreen compositions.

Surprisingly, the SPF boosting effect is reached by the compositions and/or functionalized zeolites of the invention without the zeolite binding to the filter, e.g., with no absorption or encapsulation. According to the present invention, in fact, the zeolite and the UV filter do not interact but simply form a physical mixture. As demonstrated by SEM images (FESEM−EDS (Supra 55VP/Zeiss+UltraMax 170 mm2/Oxford Instruments)), according to the present invention, the zeolite is not integrated and is not part of the UV filter and does not act as a carrier. As will be apparent from the following experimental section, the zeolites according to the invention have rather a booster action. This is also confirmed by the fact that without a UV filter, zeolites of the invention have no action as a solar filter.

As will be apparent to a person skilled in the art, the composition of the invention can be included in diverse items and products such as: leave-on skin lotions and creams, shampoos, hair conditioners, shower gels, toilette bars, antiperspirants, deodorants, shave creams, lipsticks, lip-balms, stick formulations, foundations, sunscreen lotions/creams and the like. It is noted that sunscreen agents are active only when exposed to UV radiations. In cases where no prevention against UV radiations/sunburn is needed, for example when the composition is used as a shower gel, applying the composition to the skin cannot be considered as a therapeutic treatment and the composition is mostly used as a cosmetic rather than a medicament.

Therefore, the present invention also refers to non-therapeutic uses, e.g., cosmetic uses, of the composition, according to any one of the embodiments disclosed herein.

Other aspects and advantages of the invention will be apparent from the experimental section below.

### Examples

Hereinafter, the invention will be explained in more detail with reference to the following non-limiting examples. Modifications or variations of the embodiments here exemplified, obvious to an expert in the art, are encompassed by the appended claims.

The comparative and inventive sunscreen compositions were prepared according to the following methods incorporating the ingredients as listed in Table 1 and 2, and the SPF compared.

### Manufacturing Process for Oil in Water Cream (B1, S1, S2, C1):

1. Weight components of phase A and heat up to 75-80° C., under stirring.
2. When the temperature is reached, add phase B and homogenize with UltraTurrax for 5 minutes at least 3500 rpm.
3. Weight components of phase C and heat up to 75-80° C., under stirring.
4. When both emulsifier and the UV filter are completely dissolved, add phase D and continue to mix until the SPF booster is fully incorporated in phase C.
5. When phases A+B and C+D are uniform and at their designated temperatures, add phases C+D to phases A+B homogenize with UltraTurrax at 4000 rpm for 10 minutes.
6. Cool down to room temperature under gentle stirring, then add one by one ingredients of Phase E.
7. Adjust the pH to 5.5-6.5 with citric acid if necessary

TABLE 1
Inventive and comparative example in oil-in water formulations

| Phase | INCI | % W/W B1 | % W/W S1 | % W/W S2 | % W/W C1 |
|---|---|---|---|---|---|
| A | Aqua | 65.2 | 63.2 | 63.2 | 63.2 |
| | Glycerin | 5 | 5 | 5 | 5 |
| | Tetrasodium Glutamate | 0.1 | 0.1 | 0.1 | 0.1 |
| | Diacetate | | | | |
| B | Xanthan Gum | 0.5 | 0.5 | 0.5 | 0.5 |
| C | Cetearyl Alchol | 3 | 3 | 3 | 3 |
| | Glyceryl Stearate | 4 | 4 | 4 | 4 |
| | Caprylic/Capryc Triglyceride | 3.8 | 3.8 | 3.8 | 3.8 |
| | C12-C15 Alkyl benzoate | 4.8 | 4.8 | 4.8 | 4.8 |
| | Bis-Ethylhexyoxyphenol | 2 | 2 | 2 | 2 |
| | MethoxyPhenyl Triazine | | | | |
| | Butyl | 2.5 | 2.5 | 2.5 | 2.5 |
| | Methoxydibenzoylmethane | | | | |
| | Ethylhexyl methoxycinnamate | 7 | 7 | 7 | 7 |
| D | SPF booster ZSM-5/NH4+ | — | 2 | — | — |
| | zeolite functionalized with | | | | |
| | thiethoxycaprylyl silane | | | | |
| | SPF booster ZSM-5/NH4+ | — | — | 5 | — |
| | zeolite functionalized with | | | | |
| | thiethoxycaprylyl silane | | | | |
| | SPF booster ZSM-5/NH4+ | — | — | — | 2 |
| | zeolite | | | | |
| E | Phenylpropanol and | 1 | 1 | 1 | 1 |
| | propanediol and caprylyl glycol | | | | |
| | and Tocopherol | | | | |
| | Tocopheryl acetate | 0.4 | 0.4 | 0.4 | 0.4 |
| | Fragrance | 0.5 | 0.5 | 0.5 | 0.5 |
| F | Citric Acid, solution in water | 0.2 | 0.2 | 0.2 | 0.2 |
| | 30% | | | | |

### Manufacturing Process Stick Formulation (B2, S3):

1. Combine and mix together the reagents of phase A and heat up at 80-85° C., until well melted and homogenous.
2. Separately, blend together all the reagents of phase B and heat up at 80-85° C., until well melted and homogenous.
3. Add phase B to phase A and stir until homogenous.
4. Take it off the heat and mix with prior-blended phase C.
5. Pour off while still molten.

TABLE 2
Inventive and comparative example in stick formulations

| | | % W/W | % W/W |
|---|---|---|---|
| Phase | INCI | B2 | S3 |
| A | Caprylic capric triglicerides | 4.11 | 3.99 |
| | Isoamyl laurate | 15.75 | 15.30 |
| | C12-C15 Alkyl benzoate | 9.75 | 9.45 |
| | Caprylic/Capric Triglyceride (and) | 50.71 | 49.20 |
| | Polyurethane-79 | | |
| | SPF booster ZSM-5/NH₄⁺ functionalized | 0 | 3.00 |
| | with thiethoxycaprylyl silane | | |
| B | Diethylamino hydroxybenzoyl hexyl | 6.70 | 6.50 |
| | benzoate | | |
| | Bis-Ethylhexyloxyphenol | 9.78 | 9.49 |
| | Methoxyphenyl Triazine | | |
| C | Zea Mays Germ Oil, Daucus carota | 1.37 | 1.33 |
| | sativa Root Extract | | |
| | Methyl Glucose Dioleate | 1.22 | 1.18 |
| | Parfum | 0.61 | 0.59 |

### Manufacturing Process Stick Formulation (B3, S4, S5, C2, C3):

1. Combine and mix together the reagents of phase A and heat up at 80-85° C., until well melted and homogenous.
2. Separately, blend together all the reagents of phase B and heat up at 80-85° C., until well melted and homogenous.
3. Add phase B to phase A and stir until homogenous.
4. Take it off the heat and mix with prior-blended phase C.
5. Pour off while still molten.

TABLE 3
Inventive and comparative example in stick formulations

| | | % W/W | % W/W | % W/W | % W/W | % W/W |
|---|---|---|---|---|---|---|
| Phase | INCI | B3 | S4 | S5 | C2 | C3 |
| A | Caprylic capric | 4.11 | 3.99 | 3.99 | 3.99 | 3.99 |
| | triglicerides | | | | | |
| | Isoamyl laurate | 15.75 | 15.30 | 15.30 | 15.30 | 15.30 |
| | C12-C15 Alkyl benzoate | 9.75 | 9.45 | 9.45 | 9.45 | 9.45 |
| | Caprylic/Capric | 50.71 | 49.20 | 49.20 | 49.20 | 49.20 |
| | Triglyceride (and) | | | | | |
| | Polyurethane-79 | | | | | |
| | SPF booster BETA | 0 | 3.00 | 0 | 0 | 0 |
| | 23.8/NH₄⁺ functionalized | | | | | |
| | with silane | | | | | |
| | SPF booster BETA 307/H⁺ | 0 | 0 | 3.00 | 0 | 0 |
| | functionalized with | | | | | |
| | silane | | | | | |
| | SPF booster BETA | 0 | 0 | 0 | 3.00 | 0 |
| | 23.8/NH₄⁺ | | | | | |
| | SPF booster BETA 307/H⁺ | 0 | 0 | 0 | 0 | 3.00 |
| B | Diethylamino | 6.70 | 6.50 | 6.50 | 6.50 | 6.50 |
| | hydroxybenzoyl hexyl | | | | | |
| | benzoate | | | | | |
| | Bis-Ethylhexyloxyphenol | 9.78 | 9.49 | 9.49 | 9.49 | 9.49 |
| | Methoxyphenyl Triazine | | | | | |
| C | Zea Mays Germ Oil, | 1.37 | 1.33 | 1.33 | 1.33 | 1.33 |
| | Daucus carota sativa | | | | | |
| | Root Extract | | | | | |
| | Methyl Glucose Dioleate | 1.22 | 1.18 | 1.18 | 1.18 | 1.18 |
| | Parfum | 0.61 | 0.59 | 0.59 | 0.59 | 0.59 |

### SPF In Vitro Evaluation

It has been developed an internal full in-vitro method to calculate the protection factor (SPF_{in vitro}) of a sunprotection product against erythema-inducing radiation calculated with a calibration based on a set of sunprotection standards.

The method is formulated according to the principles recommended (Protocol named “In vitro SPF Double Plate method) by the European Cosmetics and Perfumery association (former Colipa, then Cosmetics Europe) in 2011 and internally modified. By consequence, the calculated protection factor is an internal value used to compare the samples and may be not directly comparable with fully regulated in vivo tests.

The test is based on the assessment of UV-transmittance through a thin film of sunscreen sample spread on a roughened PMMA (Poly (methyl-methacrylate)) substrate. In the inspiring procedure, exposure to a radiation from an UV exposure source is required. UV exposure is not used in this method; each set of sunscreen transmission data is mathematically corrected so that the in vitro SPF data yield the same SPF values provided by several certified SPF standards.

A Perkin-Elmer LAMBDA 1050+ UV/Vis/NIR spectrophotometer equipped with a 150 mm Perkin-Elmer LAMBDA integrating sphere is used to measure the absorbance A properties of the sunscreen on the test plates and absorbance values are used to calculate SPF through the following integral formula:

$${SPF}_{in vitro} = \frac{{\int }_{290 nm}^{400 nm} E ( λ ) \times I ( λ ) d λ}{{\int }_{290 nm}^{400 nm} E ( λ ) \times I ( λ ) \times 1 {0}^{- C \times A ( λ )} d λ}$$

Where:
E: tabled CIE-1987 erythema action spectrum;
I: tabled Midday mid-summer global irradiance at 40° N;
A: measured absorbance;
C: correction coefficient

The UV spectrophotometer wavelength range shall span the primary waveband of 290 to 400 nm, covering both the ranges 290-320 nm (UV-B) and 320-400 nm (UV-A) required in SPF calculation.

A correction coefficient such that SPF_{in vitro}=SPF_{i, standard}, i=P1, P2, P3 (certified standards in different ranges of sunprotection, see table herebelow for certified values) is used in the calculation.

Mean and Acceptance limits of SPF reference
formulations - Compliance ISO 24444: 2019

| formulation | mean | Lower Limit | Upper Limit |
|---|---|---|---|
| P2 | 16.,1 | 13.7 | 18.5 |
| P5 | 30.6 | 23.7 | 37.4 |
| P8 | 63.1 | 43.9 | 82.3 |

The UV spectrophotometer wavelength range shall span the primary waveband of 290 to 400 nm, covering both the ranges 290-320 nm (UV-B) and 320-400 nm (UV-A).

In the following table it is reported a mean SPF boosting value (%), calculated as:

$$mean SPF boosting ( \% ) = \frac{[ ( {SPF}_{B} - {SPF}_{P} ) - ( {SPF}_{F} - {SPF}_{P} ) ]}{{SPF}_{F} - {SPF}_{P}} \times 100$$

Where:
SPF_{B}: measured SPF of sample formulation with UV filter plus UV booster
SPF_{F}: measured SPF of sample formulation with UV filter
SPF_{P}: measured SPF of placebo formulation (without UV filter and UV booster)

The in-vitro results for both oil-in-water and stick formulations are reported in the following table.

TABLE 4

| Ref. | Formulation | Ingredient type as SPF booster | SPF value ± StDev | % SPF boosting |
|---|---|---|---|---|
| B1 | Cream | no | 13.5 ± 1.2 | Negligible |
| S1 | Cream | ZSM-5/NH4+ functionalized | 30.5 ± 0.7 | 126 |
| | | with silane | | |
| S2 | Cream | ZSM-5/NH4+ functionalized | 57.5 ± 9.3 | 326 |
| | | with silane | | |
| C1 | Cream | ZSM-5/NH4+ | 23.1 ± 9.9 | 71 |
| B2 | Stick | no | 87 ± 9 | Negligible |
| S3 | Stick | ZSM-5/NH4+ functionalized | 125 ± 19 | 44 |
| | | with silane | | |
| B3 | Stick | no | 26.4 ± 3.9 | Negligible |
| S4 | Stick | SPF booster BETA 23.8/NH4+ | 135.6 ± 37.4 | 413 |
| | | functionalized with silane | | |
| S5 | Stick | SPF booster BETA 307/H+ | 86.4 ± 22.9 | 227 |
| | | functionalized with silane | | |
| C2 | Stick | SPF booster BETA 23.8/NH4+ | 60.1 ± 9.8 | 128 |
| C3 | Stick | SPF booster BETA 307/H+ | 52.6 ± 11.8 | 99 |

Table 4, clearly shows that the presence of an SPF booster based on zeolites according to the present invention, and specifically functionalized with silane moieties, contribute to significantly boost the UVB performances.

Indeed, when a ZSM-5 type zeolite functionalized with a thiethoxycaprylyl silane is employed as SPF booster in both stick and cream formulations (see S1-S3), the UVB performance has an increase higher than 40% with respect to the reference compositions which do not contain any SPF booster systems. Also, when a BETA type zeolite functionalized with a thiethoxycaprylyl silane is employed as SPF booster in a stick formulation (see S4-S5), the UVB performance has an increase higher than 200% with respect to the reference compositions (B3) which do not contain any SPF booster systems.

On the contrary, as disclosed in counterexample C1-C3, the same zeolites particles which do not have the silane functionalization do not reach the same boosting effect.

1. A sunscreen composition comprising:
a) at least one UV filter; and
b) zeolites as SPF boosters,
wherein said zeolites are selected in a group consisting of Beta type, ZSM-5 type (MFI), Linde type (LTA), Mordenite type (MOR) and Faujasite type (FAU) functionalized with a silane moiety selected among thiethoxycaprylyl silane Stearyl Triethoxysilane, Trihydroxymethylsilane or Methoxypolyethyleneoxypropyltrymetoxysilane.

2. A composition according to claim 1, wherein the zeolites comprise ammonium or hydrogen as counter-ion at an ion exchange site of the framework structure.

3. A composition according to claim 1, wherein the particle size distribution is comprised between 0.1 and 15 μm.

4. A composition according to claim 1, wherein the amount of zeolite is comprised between 0.1 and 5.0% with respect to the total weight of the composition.

5. A composition according to claim 1, wherein the amount of silane is comprised between 0.2 and 10.0% with respect the total weight of the zeolite.

6. A composition according to claim 1, wherein the UV filter is an organic UV filter in an amount comprised between 1 and 15% wt with respect to the total amount of the composition.

7. A composition according claim 1, wherein the UV filter is an organic molecule selected in a group consisting of Octocrylene, Ethylhexyl Salicylate, Homosalate, Butyl Methoxydibenzoylmethane, Diethylamino hydroxybenzoyl hexyl benzoate, Bis-Ethylhexyloxyphenol Methoxyphenyl Triazine, Ethylhexyl Triazone, Avobenzone, Diethylhexyl Butamido Triazone, Ethylhexyl Methoxycinnamate, Phenylbenzimidazole sulfonic acid, Tris-biphenyl triazine, Tris-biphenyltrazine (nano), Oxybenzone, Octinoxate, Octyltriazone, Octisalate, Padimate O, Bis-piperazine HAA299, Bis-piperazine HAA299 (nano), (2-Ethoxyethyl (2Z)-2-cyano-2-[3-(3-methoxypropylamino) cyclohex-2-en-1-ylidene] acetate), Sulisobenzone, Drometrizole trisiloxane, Methylene bis-benzotriazolyl tetramethylbutylphenol, Benzylidene camphor sulfonic acid, 4-Methylbenzylidene Camphor, Polysilicone-15, Sulisobenzone sodium, Aminobenzoic acid, PEG-25 PABA, Methylene Bis-Benzotriazolyl Tetramethylbutylphenol, Methylene Bis-Benzotriazolyl Tetramethylbutylphenol nano, Menthyl anthranilate, Isopentyl-4-methoxycinnamate, terephthalylidene dicamphor sulfonic acid, Dioxibenzone, Cinoxate, camphor benzalkonium sulfate and mixtures thereof.

8. A composition according to claim 1, wherein the UV filter is an inorganic compound in an amount comprised between 1 and 25% wt.

9. A composition according to claim 8, wherein the UV filter is an inorganic compound selected in a group consisting of Zinc Oxide, Anatase titanium dioxide, Rutile, titanium dioxide and mixtures thereof considering all size range both nano-, sub- and micro-metric.

10. A composition according to claim 1, wherein the UV filters are a mixture of organic UV filter and inorganic UV filters.

11. A composition according to claim 1, wherein the zeolites provide an SPF-boosting action to the sunscreen composition of at least 20%.

12. A composition according to claim 1, further comprising at least one of a cosmetically acceptable emollient, carrier fluid, pigment, humectant, vitamin, antioxidant, emulsifier, co-emulsifier, hydrophilic or hydrophobic thickeners, wax, butter, film former, silicone, surfactant, active agent, extract, fragrance, preservative, pH-adjusting agent, suspending agent, chelating agent.

13. A composition according to claim 1 formulated as a stick.

14. A composition according to claim 1, wherein the composition is a homogenous oil-in-water emulsion.

15. A composition according to claim 1 for use as a sunscreen agent, particularly for use in a method for protecting a keratinous surface from UV radiation comprising contacting the keratinous surface with said sunscreen composition.

16. (canceled)
